(19) United States
(12) Patent Application Publication
(10) Pub. No.: US 2016/0135889 A1
(43) Pub. Date: May 19, 2016

(54) METHOD FOR DEPILATION BY PHOTOTHERMOLYSIS WITH MELANIN

(71) Applicant: DERMOPARTNERS,S.L., Rafelbunyol-Valencia (ES)

(72) Inventors: Gabriel SERRANO SANMIGUEL, Valencia (ES); Juan Manuel SERRANO NUÑEZ, Valencia (ES); Gabriel SERRANO NUÑEZ, Valencia (ES)

(21) Appl. No.: 14/787,100

(22) PCT Filed: May 7, 2013
(86) PCT No.: PCT/ES2013/070088

§ 371 Date: Jan. 21, 2016

(51) Int. Cl. A61B 18/20; A61Q 9/04; A61K 8/72

(52) U.S. Cl. CPC A61B 18/203; A61K 8/72; A61Q 9/04; A61B 2018/00476

(57) ABSTRACT

Improved melanin-enhanced photothermolysis hair removal method, which includes the application of liposome-encapsulated melanin solutions as a chromophore onto the skin and the use of mechanical devices having microneedles to favor absorption thereof by the hair follicles.

### OBJECT OF THE INVENTION

The proposed invention relates to a new improved melanin-enhanced photothermolysis hair removal method for white, blond or gray hair applicable to the beauty and photoepilation salon industry.

### BACKGROUND OF THE INVENTION

The fundamental principle of laser hair removal is selective photothermolysis (SPTL), the coincidence of a specific wavelength of light and a duration of impulses such as to obtain an optimum effect on a chosen tissue with a minimum effect on the surrounding tissue. Lasers can cause localized damage on selectively heating dark target matter, melanin, in the area that causes hair growth, the follicle, while not heating the rest of the skin. The light is absorbed by the dark objects so that the laser energy may be absorbed by dark skin matter, but with much greater speed and intensity. This dark target matter, or chromophore, may be natural or may be introduced artificially.

Melanin is considered to be the main chromophore for laser hair removal. Melanin occurs naturally in the skin and gives skin and hair their color. There are two types of melanin in hair. Eumelanin gives hair its chestnut or brown color, while pheomelanin gives hair its blond or red color. Due to the selective absorption of laser light photons, only black or chestnut hair can be removed. Laser works best with thick brown hair. Fair skin and brown hair are an ideal combination, being the most effective and producing the best results, but the new lasers are now capable of targeting dark brown hair with some success in dark-skinned patients.

The new lasers can target hair with little or no melanin by applying liposome-encapsulated artificial melanin solutions onto the skin, which is only accumulated in the hair and its follicle. In this manner, even patients with lighter-colored hair can remove unwanted hair using laser or IPL (Intense Pulsed Light) devices. The idea of selective staining of follicles of blond, white and gray hair could be an excellent approach to permanent laser hair removal.

Liposomal melanin lotions have been used in recent years with conflicting results.

In this connection, mention should be made of the pre-existence of U.S. Pat. No. 5,766,214 (Thomas L. Mehl and Nardo Zaias), where reference is made to the foregoing, through the topical application of liposome-encapsulated melanin to darken the hair follicles before subjecting the skin to photoepilation.

The main drawback of this method is that the patient must apply the melanin preparation six to eight times a day, two to five weeks prior to photoepilation, in order to allow absorption by the hair follicles. Successful hair removal was proportional to the amount of solution applied, i.e. the application of greater amounts of product leads to greater hair removal but with a higher cost.

Recently, the use of dermal puncture has been proposed as a new physical strategy for increasing the transdermal application of drugs. Since 1995, this technique has been used to achieve percutaneous collagen induction aimed at reducing skin imperfections [References 28 and 29]. To date, dermal puncture has been proposed mainly as an effective method for treating scars and wrinkles [References 30 and 31], and is carried out by rolling a special device over the skin which comprises a rotating body having a variable number of microneedles.

These types of devices are disclosed in patents WO0249711 (Horst LIEBL) and EP1764129 (Horst LIEBL).

### DESCRIPTION OF THE INVENTION

The improved melanin-enhanced photothermolysis hair removal method proposed by the invention consists of an improved method of applying artificial melanin for the permanent removal of white, gray and blond hair based on the use of light energy that is absorbed by topically applied artificial melanin granules, resulting in death of the hair-producing tissue.

This method reduces the application time for introducing artificial melanin in the hair follicle and the amount of product required to induce hair removal.

Liposome-encapsulated artificial melanin penetrates in greater amounts and more quickly through the skin using a roller device having microneedles.

After the liposomal melanin solution is applied onto the skin, the roller is made to roll over the skin. In this case the needles penetrate the skin, opening micro-channels wherethrough melanin penetrates through the epidermis until reaching the hair follicle.

In such cases, this artificial melanin in the hair follicle is the chromophore for the selective photothermolysis of unwanted non-pigmented hair. Since the channels in the skin remain open for a long time, additional melanin can penetrate through the skin. Optionally, this method can be enhanced by means of a massage or similar measures.

Depending on the frequency with which the device moves over the skin, the number of channels opened therein may be specifically controlled, in addition to the degree to which the melanin can penetrate through the treated skin. Since the opened channels close again after a few minutes, damage to the epidermis is minimal. Therefore, the initially mentioned damage related to the removal of the epidermis does not occur in the case of the invention. In any case, light reddening of the skin is observed which, however, subsides completely in a few days, in most cases in one or two days. Therefore, the skin treatment practically has no side-effects and, in general, is completely painless.

Another advantage consists of the fact that the time required for the product to penetrate through the skin (hair follicle) and also the amount of product used may be considerably reduced, since the penetration of the ingredient through the lower skin layers is highly effective. All this simplifies and cheapens the method.

### DESCRIPTION OF THE DRAWINGS

In order to supplement this specification and aid understanding thereof, the following drawings have been included:

FIG. 1. Shows images of the skin marked with a fluorescent dye to detect the location of liposome-encapsulated melanin for laser removal of white hair. A1 and B1 correspond to samples without fluorescence excitation. A2 and B2 show the exact location of the melanin inside the hair, due to which it is a good target for light absorption to achieve removal thereof.

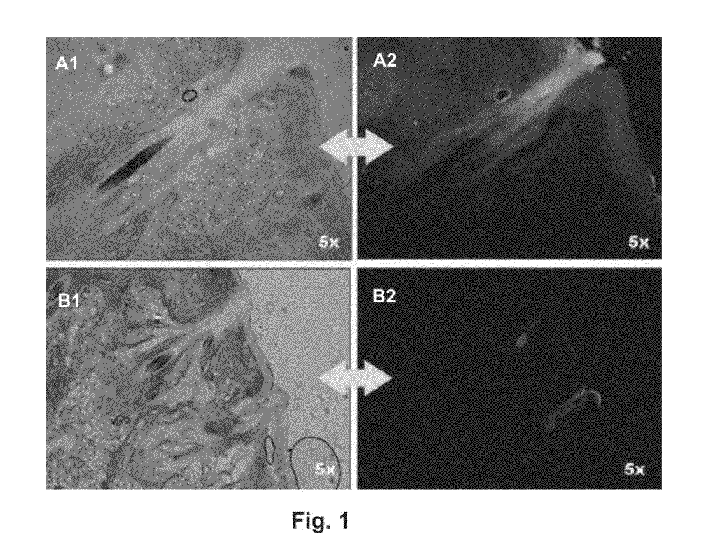

FIG. 2. Shows a sectional view of the skin, in which its different components and those of the hair follicle are shown, in addition to a representation of the absorbed melanin liposomes.

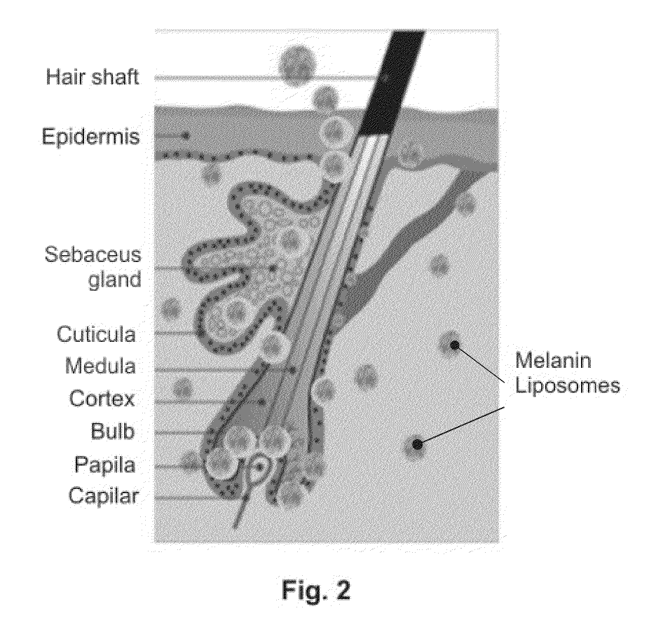

FIG. 3. Shows a representation of the manner in which to apply the microneedle roller in order to achieve homogenous dermal puncture.

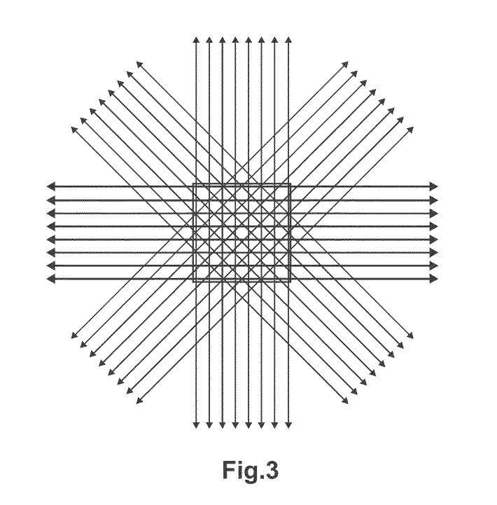

### PREFERRED EMBODIMENT OF THE INVENTION

In a preferred embodiment of the invention, the method is carried out using a liposomal sepia melanin solution, due to its resemblance to human melanin, as follows:
Disinfection of the skin before applying the method.
Shaving of the area of skin to be treated.
Washing of the skin with cleansing lotion, rubbing a cotton bud impregnated with the lotion, without using alcohol or acetone.
The melanin solution is applied at a concentration of 0.4%-0.7% onto the skin for 30 seconds, eight times in each direction, and gently massaged.
Apply the melanin solution to the microneedle roller device. Penetration of the melanin solution through the skin is achieved by rolling the device having a roller or rollers over the area to be treated. The device has one or more small paint rollers, measuring 20 mm in diameter and 20 mm in length, and the cylinder surface houses 24 circular series of eight needles each, measuring 0.5 mm in length and 0.02 mm in diameter. The tool is made to roll for 3 minutes in different directions: horizontally, vertically and diagonally, and from right to left. This ensures an even perforation pattern, resulting in approximately 250-300 perforations/cm².
The melanin solution is re-applied onto the treated area using the roller for 30 seconds, eight times in each direction, gently massaging for 1 or 2 minutes.
The treated area is left to rest for a period of 5 to 7 minutes in order to allow full absorption of the melanin.
Prior to laser exposure, the skin is washed using water and soap.
Subject the skin to be treated to laser or IPL.

### Clinical Studies

Twenty patients were treated with microneedle rollers to produce a dermal puncture every 3 minutes combined with a liposomal melanin solution on one side of the arm and only with a melanin solution on the other side, and the result was evaluated.

### Results

The side that received combined treatment (dermal puncture+melanin solution) showed a statistically significant reduction in comparison with the side treated only with a melanin solution and the clinical symptoms improved significantly.

### Conclusions

This study suggests the potential use of combining dermal puncture with rollers and microneedles with a liposomal melanin solution to achieve better results in laser hair removal treatments for white, gray and blond hair.

### Histology: Two Patients

Granular clusters of a brownish color in the stratum basale at intracellular level. These brownish-black deposits can be observed scattered throughout the papillary dermis. The post-laser biopsy reveals microvascular lesions (areas with extravasation of blood from capillaries) in the papillary dermis. Differences with regard to application with a roller and microneedles and without a roller can be observed. With a roller and microneedles the pigment is deeper and reaches the papillary dermis.

### REFERENCES

Hoffman R M. Topical liposome delivery of molecules to hair follicles in mice. J Dermatol Sci. 1997; 14(2):101-8.
Hoffman R M. Topical liposome targeting of dyes, melanins, genes, and proteins selectively to hair follicles. J Drug Target. 1998; 5: 67-74.
Tierney E P, Goldberg D J. Laser hair removal pearls. J Cosmet Laser Ther. 2008; 10(1): 17-23.
Leeuw de Jaap M D, Beek Nick van der M D & Neugebauer D. Permanent hair removal of white, grey and light blond hair after laser treatment combined with melanin encapsulated liposomes (Lipoxome R)
Sand M, Bechara F G, Sand D, Altmeyer P, Hoffmann K. A randomized, controlled, double-blind study evaluating melanin-encapsulated liposomes as a chromophore for laser hair removal of blond, white, and gray hair. Ann Plast Surg. 200; 58: 551-554.

De Leeuw J, De Viljder H C, Bjerring P, Neumann H A. Liposomes in dermatology today. J Eur Acad Dermatol Venereol 2009; 23(5):505-16.
A. R. Denet, R. Vanbever, and V. Pr'eat, “Skin electroporation for transdermal and topical delivery,” Advanced Drug Delivery Reviews 2004; 56: 659-674.
S. W. Hui, “Overview of drug delivery and alternative methods to electroporation”, Methods in Molecular Biology 2008; 423: 91-107.
Henry S, McAllister D V, Allen M G, Prausnitz M R. Microfabricated microneedles: a novel approach to transdermal drug delivery. J Pharm Sci. 1998; 87(8):922-5.
Haripriya Kalluri,1 Chandra Sekhar Kolli,2 and Ajay K. Banga. Characterization of Microchannels Created by Metal Microneedles: Formation and Closure. The AAPS Journal 2011; 13: 473-481.
M J. Hadgraft, “Skin, the final frontier,” International Journal of Pharmaceutics 2001; 224: 1-18.
H. Trommer and R. H. H. Neubert, “Overcoming the stratum corneum: the modulation of skin penetration. A review,” Skin Pharmacology and Physiology 2006; 19: 106-121.
A. L. Teo, C. Shearwood, K. C. Ng, J. Lu, and S. Moochhala, “Transdermal microneedles for drug delivery applications,” Materials Science and Engineering B, vol. 132, no. 1-2, pp. 151-154, 2006.
S. Henry, D. V. McAllister, M. G. Allen, and M. R. Prausnitz, “Microfabricated microneedles: a novel approach to transdermal drug delivery,” Journal of Pharmaceutical Sciences 1998; 87: 922-925.
Doddaballapur S. Microneedling with dermaroller. J Cutan Aesthet Surg 2009; 2:110-1
D. S. Orentreich and N. Orentreich, “Subcutaneous incisionless (subcision) surgery for the correction of depressed scars and wrinkles,” Dermatologic Surgery, vol. 21, no. 6, pp. 543-549, 1995.
[29] Fernandes, “Minimally invasive percutaneous collagen induction,” Oral and Maxillofacial Surgery Clinics of North America, vol. 17, no. 1, pp. 51-63, 2005.
Orentreich D S, Orentreich N. Subcutaneous incisionless (subcision) surgery for the correction of depressed scars and wrinkles. Dermatol Surg 1995; 21:6543-9.
Camirand A, Doucet J. Needle dermabrasion. Aesthet Plast Surg 1997; 21:48-51.
Fernandes D. Minimally invasive percutaneous collagen induction. Oral Maxillofac Surg Clin North Am 2006; 17:51-63.
Aust M C, Fernandes D, Kolokythas P, Kaplan H M, Vogt P M. Percutaneous collagen induction therapy: An alternative treatment for scars, wrinkles and skin laxity. Plast Reconstr Surg 2008; 21:1421-9.
Fernandes D, Signorini M. Combating photoaging with percutaneous collagen induction. Clin Dermatol 2008; 26:192-9. [PUBMED] [FULLTEXT]
Aust M C, Reimers K, Repenning C, Stahl F, j0 ahn S, Guggenheim M, et al. Percutaneous collagen induction: Minimally invasive skin rejuvenation without risk of hyperpigmentation—fact or fiction. Plast Reconstr Surg 2008; 122:1553-63.

[30] G. Fabbrocini, N. Fardella, A. Monfrecola, I. Proietti, and D. Innocenzi, “Acne scarring treatment using skin needling,” Clinical and Experimental Dermatology, vol. 34, no. 8, pp. 874-879, 2009.
Haq M I, Smith E, John D N, Kalavala M, Edwards C, Anstey A, et al. Clinical administration of microneedles: skin puncture, pain and sensation. Biomed Microdevices. 2009; 11(1):35-47.
Gill H S, Denson D D, Burris B A, Prausnitz M R. Effect of microneedle design on pain in human volunteers. Clin J Pain. 2008; 24(7):585-94.
[31] G. Fabbrocini, M. P. De Padova, V. De Vita, N. Fardella, F. Pastore, and A. Tosti, “Trattamento de ruga periorbitais por terapia de inducao de colageno,” Surgical and Cosmetic Dermatology, vol. 1, no. 3, pp. 106-111, 2009.
Mario Magarelli, Paolo Passamontil, Carlo Renieri. Purification, characterization and analysis of sepia melanin from commercial sepia ink (Sepia Officinalis). Revista CES Medicina Veterinaria y Zootecnia 2010; 5 (2): 18-28.
Sand M, Bechara F G, Sand D, Altmeyer P, Hoffmann K. A randomized, controlled, double-blind study evaluating melanin-encapsulated liposomes as a chromophore for laser hair removal of blond, white, and gray hair. Ann Plast Surg. 200; 58: 551-554.
Nanni C A, Alster T S. A practical review of laser-assisted hair removalusing the Q-switched Nd:YAG, long-pulsed ruby, and long-pulsed alexandrite lasers. Dermatol Surg. 1998; 24:1399-405.
Olsen E A. Methods of hair removal. J Am Acad Dermatol 1999; 40:143-154.
Dierickx C C, Grossman M C, Farinelli W A, et al. Permanent hair removal by normal-mode ruby laser. Arch Dermatol 1998; 134:8 37-842.
Cotsa relis G, Sun T T, Lavker R M. Label-retaining cells reside in the bulge area of pilosebaceous unit: implications for follicular stem cells, hair cycle and skin carcinogenesis. Cell 1990; 61:1329-1337.

1. An improved melanin-enhanced photothermolysis hair removal method for photoepilation of an area of skin having white, gray or blond hair, comprising the steps of:
applying a melanin solution onto the area of using a device having rollers and microneedles; and
subjecting the area of skin to laser photoepilation.

2. The improved melanin-enhanced photothermolysis hair removal method of claim 1, further comprising the steps, prior to the step of applying a melanin solution, of:
disinfecting the area;
shaving the area of skin;
cleaning the area of skin, without using alcohol or acetone.

3. The improved melanin-enhanced photothermolysis hair removal method of claim 1, in which the concentration of melanin in the melanin solution is 0.4%-0.7%.

4. The improved melanin-enhanced photothermolysis hair removal method of claim 1, in which the microneedles have a length of 0.5 mm.

5. The improved melanin-enhanced photothermolysis hair removal method of claim 1, in which the step of applying a melanin solution comprises the steps of:
applying melanin solution onto the area of skin;
gently massaging the area of skin;
applying the melanin solution onto the device having rollers and microneedles;
rolling the device having rollers and microneedles for 3 to 5 minutes over the area of skin;
applying additional melanin solution onto the area of skin;
leaving the melatonin solution on the area of skin for a period of 7 to 8 minutes; and
washing the area of skin using soap and water.

6. The improved melainin-enhanced photothermolysis hair removal method of claim 5, in which the step of rolling is performed in a plurality of directions.
